(19) United States
(12) Patent Application Publication
(10) Pub. No.: US 2007/0111298 A1
(43) Pub. Date: May 17, 2007

(54) Sample depositing device for a cell sorter

(75) Inventors: Torsten Muller, Berlin (DE); Stefan Hummel, Haseldorf (DE); Annette Pfennig, Berlin (DE)

Correspondence Address: THE LAW OFFICE OF RANDALL T. ERICKSON, P.C., 425 WEST WESLEY STREET, SUITE 1, WHEATON, IL, 60187, US

(73) Assignee: Evotec Technologies GmbH

(21) Appl. No.: 10/556,140

(22) PCT Filed: May 10, 2004
(86) PCT No.: PCT/EP04/04982

§ 371 Date: Apr. 26, 2006

(30) Foreign Application Priority Data
May 9, 2003 (DE) 103 20 871.2

(51) Int. Cl. C12M 3/00

(52) U.S. Cl. 435/287.1

(57) ABSTRACT

The invention relates to a sample depositing device, particularly for a cell sorter, comprising a sample supply (38, 39) for supplying samples to be deposited and comprising a sample storage element (36) for storing the samples. Said sample storage element (36) has a number of sample containers for storing the samples separately. The invention provides that the sample storage element (36) is mounted in a displaceable manner in order to select a sample container whereas the sample supply (38, 39) is stationary.

The invention relates to a sample depositing device, particularly for a cell sorter in accordance with the preamble of claim 1, as well as a corresponding sample depositing process in accordance with the preamble of claim 37.

A cell sorter is known from U.S. Pat. No. 5,489,506 which allows biological cells in a carrier flow to be dielectrophoretically separated, the dielectrophoretic effects used for separation being described, for example, in MÜLLER T. et al.: “A 3-D microelectrode system for handling and caging single cells and particles”, Biosensors & Bioelectronics 14 (1999) 247-256. In this, the biological cells to be sorted are sorted in accordance with a predefined sorting criterion into one of several outlet lines, the outlet lines each emerging into individual sample containers.

The disadvantage of this known sample depositing device is that the cell sorter requires a plurality of outlet lines in order to be able to convey the individual samples to the various sample containers. For this, individual suction pumps are needed in the outlet lines in the known sample depositing device.

Furthermore, a cell sorter is known which has only one outlet line, whereby in the cell sorter biological cells can be selected in accordance with a predefined selection criterion in such a way that the selected cells do not enter the outlet line. The outlet line of the cell sorter is movable over a microtiter plate serving as a sample storage element so that the samples supplied by the cell sorter can be taken to the desired sample container by way of suitable positioning of the mouth of the outlet line of the cell sorter.

Here, the cells in the outlet line are separated into fluid droplets which are directed to the sample containers of the sample storage element. The aerosol formation associated with this makes fault-free storing of the samples more difficult and is susceptible to contaminants entering and leaving the samples.

A disadvantage of this known sample depositing device with an adjustable positioning of the outlet line of the cell sorter continues to be the fact that the sorting process in the cell sorter is disrupted by the movement of the outlet line.

The task of the invention is therefore to improve the above-described known sample depositing device for a cell sorter in such a way that the sorting procedure in the cell sorter is not disrupted by the sample storage element and only desired cells from the outlet line of the cell sorter reach the sample storage element.

This task is solved on the basis of the above-described known sample depositing device in accordance with the preamble of claim 1 through the characterizing features of claim 1.

With regard to a corresponding process the task is solved by way of a sample depositing process in accordance with claim 35.

The invention is based on the technical knowledge that the movement of the outlet line of the cell sorter during positioning over the microtiter plate of the sample depositing device is associated with fluidic feedback into the cell sorter as a result of which the sorting procedures in the cell sorter are disrupted.

The invention therefore comprises the general technical teaching of arranging the outlet line of the cell sorter, and thereby the sample supply of the sample depositing device in a stationary manner in order to avoid the aforementioned disruptive fluidic feedback into the cell sorter.

On the other hand, the allocation and individual storing of each of the samples in the various sample containers of the sample storage element take place in accordance with the invention through suitable positioning of the sample storage element.

The term sample supply used within the context of the invention should be understood in a general manner and not restricted to a hose or a line, as is the case in the preferred embodiment of the invention.

Furthermore, the term sample storage element used within the context of the invention should be understood in a general manner and not limited to the microtiter plate used in the preferred embodiment of the invention. Instead, other storage containers or arrangements of storage containers can be used as sample storage elements.

Preferably, however, the sample supply of the sample depositing device in accordance with the invention has a line or a hose, the mouth of which is arranged in a stationary manner above the sample storage element. As a result of gravity and a given pumping rate, the samples emerging from the hose or line flow downwards in the direction of the sample storage element into one of the sample containers whereby selection of the desired sample container takes place by way of suitable positioning of the sample storage element.

Here, it is of particular advantage if the hose is led through a guiding piece in order to direct the mouth of the hose in the direction of the sample storage element. This is sensible as otherwise the hose could assume an undesirable spatial orientation as a result of its own elasticity so that the sample emerging from the house would possibly not enter the sample storage element. This guiding of the hose through the guiding piece is particularly advantageous if the sample storage element is located in a room (e.g. an incubator) which must be kept as sterile as possible, as the hose can then be fed into the sterile room without the need to reach into the sterile room which would be associated with the undesirable introduction of germs.

The guiding of the hose can, for example, take place by means of a groove in the guiding piece into which the hose can be inserted in order to define the course of the hose and to direct the mouth of the hose toward the sample storage element.

Preferably the groove has projections on the edges of the groove which firmly clamp the hose in the inserted condition, thereby preventing the hose from slipping out during operation of the sample depositing device.

Alternatively it is however also possible for the hose to be pressure fitted into the groove so that the hose is frictionally fixed in the groove.

Preferably the guiding piece for the hose can be autoclaved in order to allow multiple sterilization. As a material for manufacturing the guiding piece PEEK is therefore particularly suitable, but in principle the guiding piece can also be made of other materials.

In the preferred embodiment of the invention the hose and/or the guiding piece with the hose is detachably mounted above the sample storage element. This detachable mounting of the guiding piece and/or the hose above the sample storage device advantageously allows simple assembly as well as rapid replacement of the hose as the hose can be inserted into the guiding piece outside the sample depositing device.

Detachable assembly of the hose or the guiding piece with the hose can take place, for example, by way of a holding magnet which fixes the hose or the guiding piece with the hose in a detachable manner above the sample storage element. One possibility for this is the holding magnet being attached to the guiding piece, while a corresponding magnetizable holding element or another holding magnet is arranged in a stationary manner to the sample depositing device. Preferably, however, a magnetizable holding element is attached to the guiding piece while the corresponding holding magnet is mounted in a stationary manner on the sample depositing device.

Preferably the magnetizable holding element is cast into the guiding piece here or the guiding piece is injection molded around it in order to avoid corrosion of the magnetizable holding element. This makes sense as magnetizable steels often exhibit poor corrosion resistance. The corrosion-proof arrangement of the magnetizable holding element in the guiding piece therefore broadens the design scope when selecting the material for the magnetizable holding element.

It has already been set out above that the allocation of the samples to be deposited to the individual sample containers of the sample storage element takes place by way of suitable positioning of the sample storage element. Preferably therefore an actuator is provided in order to position the sample storage element accordingly. Such an actuator can be, for example, an electric motor or a pneumatic actuator, but with regard to the technical principle of the actuator for positioning the sample storage element the invention is not limited to the above types of actuator.

Preferably, however, the actuator allows positioning of the sample storage element in at least two directions in space, which are preferably in a horizontal plane.

Furthermore, it is advantageous if the sample storage device can also be moved in the direction of the sample supply and/or the mouth of the supply hose. During the storing of the samples, the sample storage device is then preferably moved so far in the direction of the sample supply that the sample supply immerses in the fluid present in the sample storage element. In the case of a hose forming the sample supply, the sample storage element is thus preferably moved so far upwards until the mouth of the hose is immersed in the fluid present in the sample storage device. This immersion during the storing of the samples is advantageous as it prevents droplet formation with subsequent droplet detachment, thereby increasing the contamination security of the sample depositing device and minimizing any possible errors. Droplet detachment in the sample supply is also disruptive as it causes flow-dynamic feedback which works its way via the sample supply back to the cell sorter and disrupts the sorting procedures there. The sample storage element is preferably also positionable in three directions in space, whereby horizontal positioning serves to select the sample container, while vertical positioning immerses the sample supply in the sample storage element in order to prevent disruptive droplet formation and detachment.

However, the disruptive droplet detachment on the sample supply can also be prevented by the distance between the sample supply and the sample storage element located below it being smaller than a material-dependent droplet detachment size. In this variant, certainly a droplet can initially form on the sample supply, this is, however, harmless as long as the droplet does not detach from the sample supply. As of a certain size the droplet comes into contact with the sample storage element or is immersed in the sample storage element, which results in controlled release of the droplet without disruptive flow-dynamic effects. The droplet detachment size depends here on the sample material and, in particular, the density and cohesive force of the sample material, so that the distance between the sample supply and the sample storage element should be adjusted accordingly.

It is furthermore advantageous if the entire sample storage element is arranged in an incubator, which preferably has a climate control equipment in order to adjust the temperature and/or humidity in the incubator. This is advantageous in the storing of biological samples which require a specific climatic environment.

Preferably, the incubator is operated at a slight overpressure with prefiltered, sterile air. As in clean room ventilation the overpressure prevents germs penetrating in from outside.

With CO₂ gassing of the indicator and adjusting the relative humidity, it is also possible to prevent fluid evaporation from the sample container.

To monitor the sample storage element, an inspection window can be provided in the incubator, whereby the inspection window can be opened, for example, in the form of a flap in order to allow manual access if necessary.

However, it is also possible to arrange a camera in the incubator in order to monitor the process of sample deposition. In this case, the inspection window in the incubator can be dispensed with.

The sample storage element (e.g. a microtiter plate) is preferably arranged in a detachable manner in the sample depositing device according to the invention in order to allow replacement of the sample storage element. For example, the sample storage element can be inserted in to the sample depositing device manually or by a robot. With a microtiter plate as the sample storage element, the cover of the microtiter plate should only be opened after introduction into the sample depositing device and/or the incubator in order to avoid contamination. Furthermore, with a microtiter plate as the sample storage element it is advantageous if this is guided by laterally arranged metal balls or sprung spherical pressure elements. The metal balls or sprung spherical pressure elements also guarantee an evening out of dimensional tolerances of the used microtiter plates. In some circumstances, the dimensions of the microtiter plates used differ slightly as a result of manufacturing tolerance, which is evened out in this way.

Moreover, the sample storage element (e.g. a microtiter plate) can be covered with a film which can be penetrated by the sample supply (e.g. a hose tip) in order to deposit the samples in the sample storage element. Such film covering of the sample storage element allows sterile depositing of the samples, reduces evaporation and reduces the pH shift. The film used to cover the sample storage element can be, for example, an aluminum, silicon or rubber film.

It should also be mentioned that the term sample used within the context of the invention should be understood in a general manner and is not restricted to biological cells, which are sorted by a cell sorter.

An advantage of the sample depositing device in accordance with the invention is the possibility of working with low contamination. The structure and design of the sample depositing device is space-optimized to reduce both the introduction of contaminants (e.g. germs) into the sample as well as the transfer of substances (e.g. pathogens) from the sample through the air. Furthermore, the sample depositing device in accordance with the invention is optimized to ensure rapid, careful and error-free storing of particles (e.g. biological cells) into the sample storage device or similar storage container.

Finally the invention also comprises a cell sorter, a particle manipulator and a fluidic system with a sample depositing device in accordance with the invention as has been described above.

Other advantageous further developments of the invention are characterized in the dependant claims or will be explained below together with the description of the preferred embodiments with the aid of the figures.

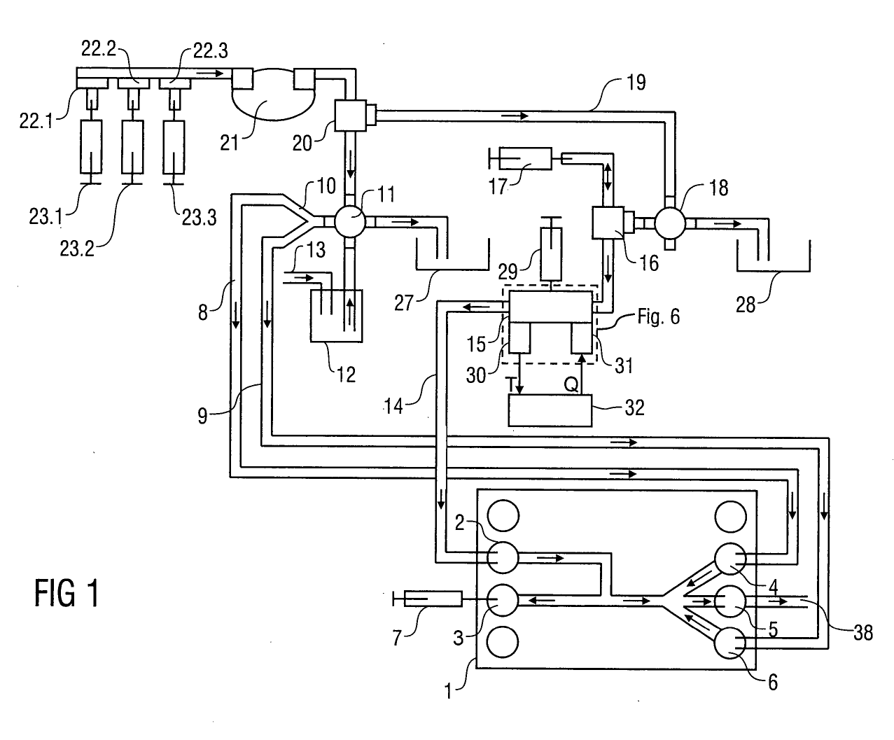

FIG. 1 shows a fluidic diagram of a cell sorter in accordance with the invention without the sample depositing device in accordance with the invention.

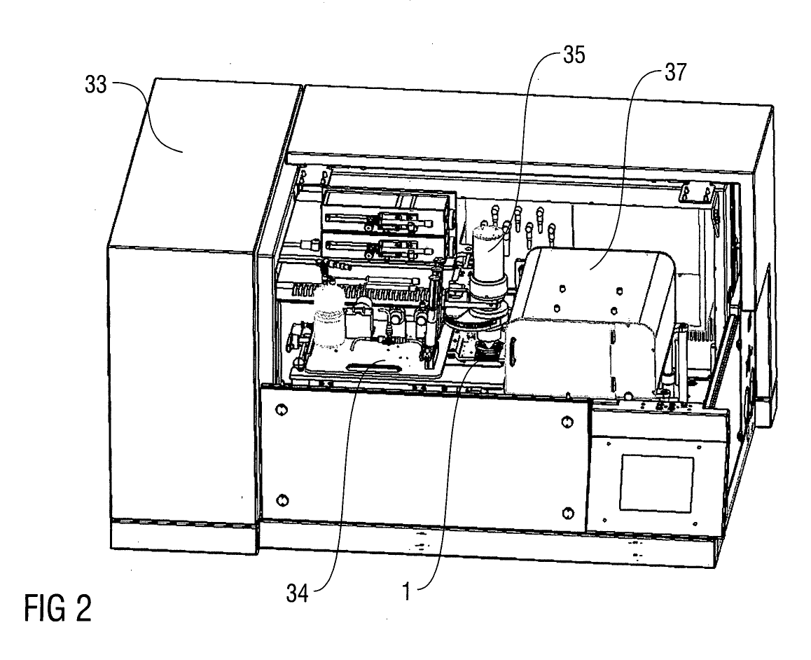

FIG. 2 shows a perspective view of the cell sorter in FIG. 1 with the sample depositing device in accordance with the invention.

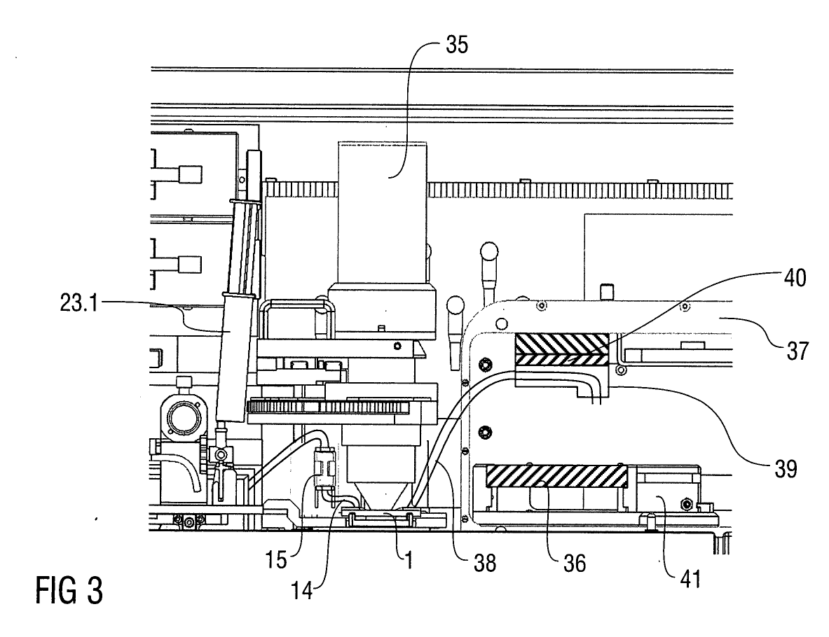

FIG. 3 shows a front view of the cell sorter in FIG. 2 in the area of the sample depositing device and

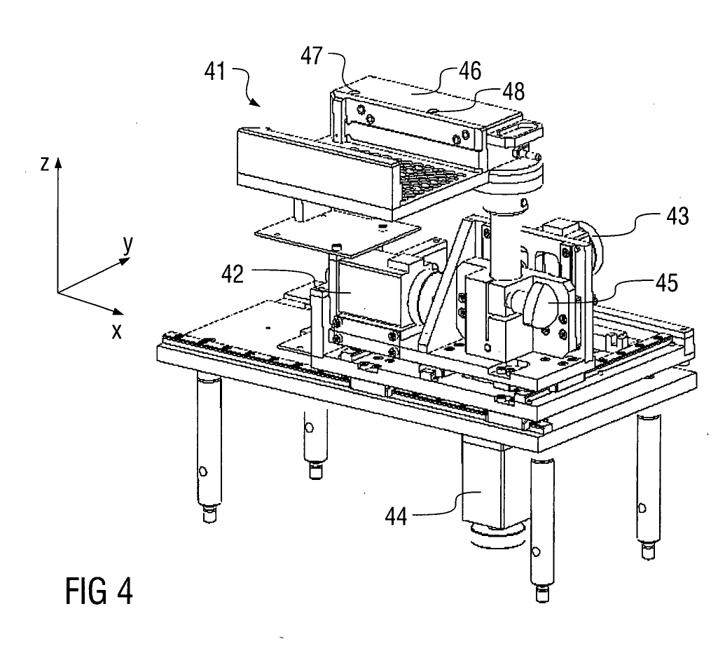

FIG. 4 shows a perspective view of the mechanics of the sample depositing device in FIG. 3.

The schematic view in FIG. 1 shows a cell sorter in accordance with the invention, which sorts biological cells dielectrophoretically by way of a microfluidic sorter chip 1, whereby the sorter chip 1 is supported in a vibration absorbing manner.

The techniques of dielectrophoretic influencing of biological cells are described, for example, in MÜLLER T et al.: “A 3-D microelectrode system for handling and caging single cells and particles”, Biosensors & Bioelectronics 14 (1999) 247-256 so that a detailed description of the dielectrophoretic processes in the sorting chip 1 is dispensed with and reference is made to the above publication in this respect.

The sorting chip 1 has several connections 2-6 for fluidic contacting, the fluidic contacting of connections 2-6 being described in DE 102 13 272, the content of which is incorporated by reference herein.

Connection 2 of the sorting chip 1 serves to take up a carrier flow with the biological cells to be sorted, whereas connection 3 of the sorting chip 1 serves to remove the selected biological cells, which are not examined further on the sorting chip 1. The selected biological cells can be collected with a suction syringe 7, which can be connected to connection 3 of the sorting chip 1. The outlet 5 of the sorting chip 1 on the other hand serves to remove the biological cells, which are of interest and which can subsequently be processed further or examined.

Connections 4 and 6 of the sorting chip 1 furthermore serve to supply a so-called sheath flow, which has the task of carrying the selected biological cells to connection 5 of the sorting chip 1. With regard to the function of the sheath flow reference is made to German patent application DE 100 05 735 so that a detailed description of the function of the sheath flow is dispensed with below.

Connections 4 and 6 of the sorting chip are connected via two sheath flow lines 8, 9, a Y-piece 10 and a four-way valve 11 to a pressurized container 12, which contains a cultivation medium for the sheath flow.

The pressurized container 12 is overpressurized by a compressed air line 13 so that with the four-way valve set appropriately, the cultivation medium in the pressurized container 12 flows via the Y-piece 10 and the sheath flow lines 8, 9 to connections 4, 6 of the sorting chip 1.

Connection 2 of the sorting chip 1 on the other hand is connected via carrier flow line 14 to a particle injector 15.

Upstream the particle injector 15 is connected via a T-piece 16 to a carrier flow injector 17, which is mechanically driven and injects a predefined fluid flow of a carrier flow.

In addition to this, the T-piece 16 is connected upstream via a further four-way valve 18 and a filling flow line 19 to a three-way valve 20. The three-way valve 20 allows cleaning of the sheath flow lines 8, 9 as well as of the carrier flow line 14 before actual operation. For this, the three-way valve 20 is connected upstream via a peristaltic pump 21 to three three-way valves 22.1-22.3 each of which is connected to an injector reservoir 23.1-23.3. The injector reservoirs 23.1-23.3 serve to supply a filling flow to clean the entire fluidic system before actual operation, whereby the injector reservoir 23.1 contains 70% ethanol while injector reservoir 23.2 contains distilled water as the filling flow substance. Injector reservoir 23.3 contains a manipulation fluid, such as, for example, a buffer solution as the filling flow substance.

The cell sorter also has a collection container 27 for excess sheath flow as well as a collection container 28 for excess filling flow.

The cleaning procedure, which is carried out before actual operation of the cell sorter in order to free the sheath flow line 8, 9, the carrier flow line 14 and the remainder of the fluidic system of the cell sorter of air bubbles and contaminants will be initially described below.

For this, the three-way valve 22.1 is first opened and ethanol is injected as filling flow from injection reservoir 23.1 whereby the ethanol is initially conveyed by the peristaltic pump 21 to the three-way valve 20. During this cleaning procedure the three-way valve 20 is set in such a way that a part of the filling flow conveyed by the peristaltic pump 21 is conveyed further via the filling flow line 19, while the remainder of the filling flow conveyed by the peristaltic pump 21 reaches the four-way valve 11. The two four-way valves 11, 18 are in turn set in such a way that the filling flow is conveyed through the sheath flow lines 8, 9 and the carrier flow line 14. Cultivation medium also flows from the pressurized container 12 into the collection container 27 in order to flood the lines briefly.

After cleaning the cell sorter with ethanol as described above, cleaning with distilled water and/or manipulation fluid, such as, for example, a buffer solution, takes place in the same manner whereby the three-way valves or 22.2 or 22.3 respectively are opened.

During the above cleaning procedure excess filling flow can be discharged from the four-way valve 18 into the collection container 28.

After the cleaning procedure the three-way valves 22.1-22.3 are closed and the peristaltic pump 21 is switched off.

To start the sorting operation, the four-way valve 11 is set in such a way that the pressurized container 12 is connected to the Y-piece 10 so that the manipulation fluid (e.g. a cultivation medium) contained in the pressurized container 12 is forced into the sheath flow line 8, 9 by the overpressure in the pressurized container 12.

Furthermore, during the sorting operation the four-way valve 18 is set in such a way that there is no flow connection between the T-piece 16 and the four-way valve 18.

The carrier flow injected by the carrier flow injector 17 then flows via the T-piece 16 into the particle injector 15, whereby biological cells are injected into the carrier flow by a further injector 29. The carrier flow with the injected biological cells then flows from the particle injector 15 via the carrier flow line 14 to connection 2 of the sorting chip.

It should furthermore be mentioned that a temperature sensor 30 is attached to the particle injector 15 in order to measure the temperature T of the particle injector 15.

In addition, there is a temperature control element 31 in the form of a Peltier element on the particle injector 15 in order to be able to heat or cool the particle injector 15.

The heating and cooling energy Q is predefined by a temperature controller 32, which is connected to the temperature sensor 30 on the inlet side and adjusts the temperature T of the particle injector 15 to a predetermined target value.

The perspective view in FIG. 2 of the cell sorter shown in FIG. 1 will now be described below.

The cell sorter is accommodated in a housing 33 of synthetic material, the housing having a transparent cover in order to allow visual monitoring of the operation of the cell sorter.

In the housing 33 there is a structure with a so-called docking station 34 into which the essential fluidic components as well as the electrical feed lines of the cell sorter on a main board can be inserted. The docking station 34 of the cell sorter thus advantageously permits rapid and simple replacement of the essential components of the cell sorter.

The structure of the cell sorter furthermore holds the sorting chip 1, whereby a transillumination equipment 35 is arranged above the sorting chip 1 in order to illuminate the carrier flow with the particles suspended in it as it flows through the sorting chip 1.

In the drawing, on the right next to the sorting chip 1, a sample depositing device is arranged, which has a microtiter plate 36 as the sample storage element.

Here, the sample depositing device with the microtiter plate 36 is arranged in an incubator 37, the incubator 37 having a climate control equipment for controlling the temperature, humidity and/or the CO₂ gassing in the incubator 37. Climate conditioning in the incubator 37 is important so that biological samples deposited in the microtiter plate 36 also remain undamaged sterile during temporary intermediate storage in the microtiter plate 36. In addition to this, the climate control equipment produces a slight overpressure in the incubator 37 so that no particles can enter the incubator 37 from outside as this could lead to contamination. The climate control equipment furthermore filters the air entering the incubator 37 and thereby also prevents contamination of the samples.

On its front side the incubator 37 has a flap of transparent synthetic material, which forms an inspection window allowing visual monitoring of the sample depositing device.

In addition to this, a small camera is arranged in the incubator 37 in order to be able to monitor the storing of the samples and, in particular, the immersion of a hose 38 into the sample container of the microtiter plate 36, the camera not being shown for the sake of simplification. Preferably, additional LED illumination can be provided.

The hose 38 is made of Teflon here but the hose 38 can alternatively consist of PE capillaries, other synthetic materials or glass.

The structure of the sample depositing device in accordance with the invention will now be described below with reference to FIGS. 3 and 4.

The hose 38 is connected to connection 5 of the sorting chip 1 whereby the fluidic contacting of the sorting chip 1 by the hose 38 is described in DE 102 13 272, the content of which should be added to this description, so that a detailed description of the fluidic contacting of the sorting chip 1 by the hose 38 can be dispensed with here.

The hose 38 is fed into the inside of incubator 37 through a lateral, sealed opening in the incubator 37, the mouth of the hose 38 being arranged above the microtiter plate 36 and directed towards the microtiter plate 36. Through the sealing of the lateral opening and the slight overpressure in the incubator 37 the sterility is maintained here. The orientation of the hose 38 is achieved by a guiding piece 39 which has a lateral groove into which the hose 38 is pressed so that the course of the groove determines the orientation of the hose 38 above the microtiter plate 36. Projections formed in one piece are molded onto the flanks of the groove in the guiding piece 39 to prevent the hose 38 from slipping out of the groove of the guiding piece 39.

It should also be mentioned that the end of the hose 38 in the guiding piece 39 is attached in a stationary manner within the sample depositing device. This provides the advantage that no disruptive fluidic feedbacks via the hose 38 into the sorting chip 1 occur so that the sorting procedures in the sorting chip 1 can take place undisturbed.

The guiding piece 39 is fixed in a stationary manner in the hose 38 by way of a holding magnet 40, which is attached to the inner wall of the incubator 37.

The holding magnet 40 interacts with a holding element consisting of a magnetizable material and being incorporated into the guiding piece 39.

Attaching the guiding piece 39 with the hose 38 to the holding magnet 40 advantageously allows simple, germ-free assembly.

In the illustrated embodiment, the guiding piece 39 is made of PEEK and is thus autoclavable, so that a sterilization of guiding piece 39 is possible.

Via the hose 38 the samples released by the cell sorter can be placed in the various sample containers of the microtiter plate 36. Here, the desired sample container of the microtiter plate 36 is selected through appropriate positioning of the microtiter plate 36 relative to the mouth of the hose 38, which is the purpose of the mechanism shown in FIG. 4.

In addition, undesired cells or cleaning fluid can be conveyed to a collection container, which is located directly next to the microtiter plate 36.

Thus, the microtiter plate 36 is inserted into a receiving element 41, the receiving element 41 being positionable in the x, y and z direction by way of three electric motors 42-44.

In addition, the receiving element 41 can be removed from the sample depositing device by loosening a knurled screw 45. However, alternatively it is possible for the receiving element 41 to be retained by a notch. The receiving element 41 is then removed from the sample depositing device by pulling out the receiving element past the notch point.

It should also be mentioned that the receiving element 41 has a removable collection container 46 (‘waste container’) arranged next to the microtiter plate 36 into which samples can be placed, which are of no further interest and are not to be deposited in the microtiter plate 36.

The collection container 46 is made of an autoclavable material and has a removable cover on its upper side.

In addition, on the upper side of the collection container 46 there are trough-shaped recesses 47, 48 in order to prevent disruptive droplet detachment during the storing of samples in the collection container 46 as will be described below.

For this, the receiving element 41 is positioned in such a way that the mouth of the hose 38 is located above one of the two recesses 47, 48.

The receiving element 41 is then moved upwards towards the mouth of the hose 38 until the hose 38 dabs the recess 47 or 48, whereby the sample located in the hose 38 detaches and enters the collection container 46. However, there is no droplet detachment so that no disruptive flow-dynamic effects occur. The receiving element is only taken so far upwards that the droplet on the mouth of the house 38 can flow off, there is, however, no contact between the hose 38 and the receiving element 41.

On the top of the collection container 46 there can be a blind hole with a volume of approximately 50 μl, which is not shown for the sake of simplification. This blind hole allows the sheath flow rate (sheath flow volume per unit of time) to be checked before each sorting process. In order to do this, the blind hole is filled with the emerging sheath flow, the time taken to fill the blind hole being measured, from which the sheath flow rate is derived.

In addition, the hose 38 has a beveled tip and a hydrophilic coating in order to prevent disruptive droplet detachment during sample depositing.

The tip of the hose 38 further allows puncturing of a film covering the microtiter plate 36. This film can consist of aluminum or silicone, for example, and ensure sterile sample depositing. In addition, the film prevents evaporation from the microtiter plate 36 and reduces pH shifting.

In this case, the tip can be produced as a separate component and mounted on the mouth of the hose 38.

To further reduce the droplet detachment size, a wetting agent can be mixed into the carrier flow to reduce the surface tension of the carrier fluid and thereby counteracting disruptive droplet formation.

Also, in order to further reduce disruptive droplet detachment, it is also possible to position the microtiter plate 36 in such a way that a droplet formed on the tip of the hose 38 laterally comes into contact with the inner wall of a sample container so that the droplet flows off via the inner wall without detaching abruptly.

In order to avoid disruptive droplet detachment it is further possible to structure hydrophilic and hydrophobic areas of the hose 38, the tip of the hose 38 and the microtiter plate 36.

The invention is not limited to the preferred example of embodiment described above. Rather a plurality of variants and derivations is possible, which also make use of the inventive concept and thus fall under the protective scope.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. Sample depositing device, particularly for a cell sorter, with
a sample supply for supplying samples to be deposited,
a sample storage element for storing the samples, whereby the sample storage element has several sample containers for the separate storage of the samples,
wherein the sample storage element is arranged in a displaceable manner in order to select a sample container, whereas the sample supply is stationary.

41. Sample depositing device according to claim 40, wherein the sample supply has a hose the mouth of which is fixed in a stationary manner over the sample storage element.

42. Sample depositing device according to claim 41, wherein the hose is led through a guiding piece in order to direct the mouth of the hose to the sample storage element.

43. Sample depositing device according to claim 42, wherein the guiding piece has a groove into which the hose can be inserted in order to define the course of the hose and to direct the mouth of the hose to the sample storage element.

44. Sample depositing device according to claim 42, wherein the guiding piece consists of an autoclavable and sterilisable material.

45. Sample depositing device according to claims 42, wherein the guiding piece is made of a material selected from a group consisting of PEEK, LEXAN and TEFLON.

46. Sample depositing device according to claim 41, wherein the hose and the guiding piece are fixed in a detachable manner above the sample storage element.

47. Sample depositing device according to claim 46, wherein the hose and the guiding piece are attached in a detachable manner above the sample storage element by way of a holding magnet.

48. Sample depositing device according to claim 47, wherein the holding magnet is fixed in a stationary manner to the sample depositing device, whereas a magnetizable holding element is attached to the guiding piece.

49. Sample depositing device according to claim 48, wherein the magnetizable holding element is cast into the guiding piece.

50. Sample depositing device according to claim 48, wherein the guiding piece is injection molded around the magnetizable holding element.

51. Sample depositing device according to claim 40, wherein to position the sample storage element relative to the sample supply an actuator is provided.

52. Sample depositing device according to claim 40, wherein the sample storage element can be positioned laterally and in the direction of the sample supply.

53. Sample depositing device according to claim 52, wherein the sample storage element can be positioned so far in the direction of the sample supply that the sample supply is immersed in one of the sample containers of the sample storage element.

54. Sample depositing device according to claim 52, wherein the sample to be deposited is fluid and has a material-dependent droplet detachment size, whereby the sample storage element can be moved so far upwards in the direction of the sample supply that the distance between the sample supply and the sample storage element is smaller than the droplet detachment size.

55. Sample depositing device according to claim 40, wherein the sample storage element is arranged in an incubator.

56. Sample depositing device according to claim 55, wherein the incubator has an inspection window in order to allow visual monitoring.

57. Sample depositing device according to claim 55, wherein the incubator has a camera in order to allow visual monitoring.

58. Sample depositing device according to claim 55, wherein the incubator has an additional lighting in order to allow visual monitoring.

59. Sample depositing device according to claim 56, wherein the incubator has climate control equipment which sets a value selected from a group consisting of the temperature, the humidity and the carbon dioxide content in the incubator.

60. Sample depositing device according to claim 40, wherein the sample storage element is a microtiter plate.

61. Sample depositing device according to claim 40, wherein the sample storage element has test strips for taking up PCR samples.

62. Sample depositing device according to claim 40, wherein the sample storage element is arranged in a detachable manner in the sample depositing device.

63. Sample depositing device according to claim 62, wherein the sample storage element can be inserted and removed manually.

64. Sample depositing device according to claim 62, wherein the sample storage element can be inserted and removed with a robot.

65. Sample depositing device according to claim 40, wherein to guide the sample storage element laterally arranged metal balls are provided.

66. Sample depositing device according to claim 40, wherein to guide the sample storage element laterally arranged sprung spherical pressure elements are provided.

67. Sample depositing device according to claim 40, wherein the sample storage element has a cover which can be opened for storing the samples and can be closed for removal and transportation of the sample storage element.

68. Sample depositing device according to claim 40, wherein the hose is connected to the guiding piece in a detachable manner.

69. Sample depositing device according to claim 40, wherein the hose is permanently attached to the guiding piece.

70. Sample depositing device according to claim 40, wherein the sample supply is arranged above the sample storage element, whereby the distance between the sample supply and the sample storage element is smaller than a material-dependent droplet detachment size in order to prevent droplet detachment during the storing of the samples.

71. Sample depositing device according to claim 40, wherein to store samples which are not of interest a collection container is provided.

72. Sample depositing device according to claim 71, wherein the collection container has a cover which can be opened.

73. Sample depositing device according to claim 71, wherein the collection container is removable.

74. Sample depositing device according to claim 71, wherein the collection container is made of an autoclavable material.

75. Sample depositing device according to claim 71, wherein the collection container has on its upper side at least one trough-shaped recess for dabbing off a sample.

76. Sample depositing device according to claim 41, wherein the hose has an oblique tip.

77. Sample depositing device according to claim 41, wherein the hose is at least in the area of its mouth made of a hydrophilic material in order to prevent droplet detachment.

78. Sample depositing device according to claim 41, wherein the hose is at least coated with a hydrophilic material in the area of its mouth in order to prevent droplet detachment.

79. Sample depositing device according to claim 40, wherein the sample storage element is covered with a film which can be penetrated by the sample supply.

80. Cell sorter with a sample depositing device in accordance with claim 40.

81. Particle manipulator with a sample depositing device according to claim 40, in particular manipulator for the fusion and portion of biological objects.

82. Fluidic system with sample depositing device according to claim 40, in particular system for mixing fluids.

83. Sample depositing process, particularly for depositing samples in a cell sorter, with the following stages:
Positioning of a sample supply relative to a sample storage element,
Release of the sample to be deposited from the sample supply in the sample storage element,
wherein the sample supply is stationary whereas the sample storage element is moved.

84. Sample depositing process according to claim 83, wherein the sample is fluid and has a material-dependent droplet detachment size, whereby for depositing the sample the sample storage element is moved so far in the direction of the sample supply that the distance between the sample supply and the sample storage element is smaller than the droplet detachment size.

85. Sample depositing process according to claim 84, wherein when releasing the sample a distance remains between the sample supply and the sample storage element.
